(19) United States
(12) Patent Application Publication
(10) Pub. No.: US 2015/0125880 A1
(43) Pub. Date: May 7, 2015

(54) METHODS FOR INDUCTION OF ANTIGEN-SPECIFIC REGULATORY T CELLS

(71) Applicants: ImCyse SA, Liège (BE); Katholieke Universiteit Leuven, Leuven (BE)

(72) Inventor: Jean-Marie Saint-Remy, Grez-Doiceau (BE)

(21) Appl. No.: 14/397,560

(22) PCT Filed: Apr. 29, 2013
(86) PCT No.: PCT/EP2013/058835

§ 371 Date: Oct. 28, 2014

Related U.S. Application Data
(60) Provisional application No. 61/640,537, filed on Apr. 30, 2012.

(51) Int. Cl. C12N 5/0783

(52) U.S. Cl. CPC C12N 5/0637; C12N 2502/1121
USPC 435/7.24; 435/373

(57) ABSTRACT

The present invention relates to methods to elicit immature antigen-presenting cells loaded with apoptotic cells or apoptotic bodies. The present invention also relates to methods of obtaining antigen-specific regulatory T cells in vitro or in vivo. Cells loaded with apoptotic bodies/cells and regulatory T cells are obtainable by inducing apoptosis of antigen-presenting cells by cytolytic CD4+ T cells. The cells are used for suppressing or preventing diseases such as autoimmune diseases, graft rejection and allergic diseases, and medicaments related thereto. Further disclosed are the use of antigen-specific regulatory T cells for suppressing or preventing diseases such as autoimmune diseases, graft rejection and allergic diseases, and medicaments related thereto. Further disclosed are populations of antigen-specific regulatory T cells obtained by said method.

### FIELD OF THE INVENTION

The present invention relates to methods of obtaining antigen-specific regulatory T cells and their use as a medicament to treat conditions such as autoimmune diseases, allergic diseases or graft rejection.

### BACKGROUND OF THE INVENTION

Regulatory T cells (Tregs), particularly Tregs expressing the transcription repressor Foxp3 (Forkhead box P3), are essential in maintaining a normal immune homeostasis. In the absence of such cells, autoimmunity rapidly develops with clinical manifestations such as diabetes mellitus and other autoimmune diseases (reviewed in Sakaguchi et al. (2012) Nature Med. 18, 54-58). Foxp3+ Tregs are actively selected in the thymus and constitute the population of cells found in peripheral blood, which stably express Foxp3. The threshold at which cells are selected in the thymus upon cognate recognition of self peptides presented by thymic epithelial cells is such that Foxp3+ cells with significant affinity are found in the peripheral blood, in contrast to effector T cells. A current view is that peripheral tolerance is maintained, inter alia, by a balance between autoantigen-specific effector cells with weak affinity and Foxp3+ cells with higher affinity, thereby providing equilibrium towards tolerance. In addition to this central selection of Tregs, cells can be converted in the periphery to express the transcription factor Foxp3. However, expression is lower than in thymus-selected population and some reversibility of the acquired phenotype has been observed.

The properties of natural Tregs, as selected in the thymus and characterized by high and stable Foxp3 expression, make them very attractive as a means to control pathologies characterized by auto-immune responses, as well as a therapeutic tool to keep unwanted responses to graft or to allergens under control, to cite just a few. However, the number of antigen-specific natural Tregs in the periphery is very low and methods to expand them in vivo or even in vitro are neither well defined nor reliable. A method by which it would be possible to selectively expand population of Tregs would carry the potential to prevent or suppress disease processes without affecting the overall capacity of the organism to mount beneficial responses.

Apoptosis, or programmed cell death, is a physiological mechanism which helps maintain tissue homeostasis (reviewed in Fuchs and Steller (2011) Cell 147, 742-758). It has been calculated that up to 10⁶ cells are destroyed by apoptosis every minute in a human body. The enormous amount of antigens liberated by cell death has to be kept under control so as to avoid eliciting immune response against self-proteins. In fact, apoptotic cells are taken up by scavenger cells, mainly immature dendritic cells, and then processed in a way to induce tolerance. Cross-presentation of antigens derived from apoptotic cells are presented in class II major histocompatibility complexes (MHC), which are known to elicit Foxp3+ Treg expansion. Thus, apoptosis of cells, which occur in the absence of inflammatory context, represents a physiological way by which regulatory T cells are expanded.

It is therefore desirable to design a method by which it would be possible to induce apoptosis of cells presenting autoantigens or antigens to which an immune response is undesirable (such as, for example, in allergic diseases or graft rejection), which would then generate apoptotic bodies, leading to expansion of antigen-specific Tregs. Non-specific immunosuppressive therapies known in the art generally lead to susceptibility to severe infections and other serious consequences, which negatively affect quality of life. As such, it would be advantageous to develop a method whereby antigen-specific Tregs are used to treat immune diseases without the undesirable effects of traditional therapies.

A general method has been described by which it is possible to elicit antigen-specific cytolytic CD4+ T cells (cCD4+ T cells) in WO2008/017517. Such cells induce apoptosis of antigen-presenting cells after cognate recognition of peptide-MHC class II complexes. Advantageously and unlike the prior art, the present invention provides for the expansion of antigen-specific Foxp3 Tregs by inducing apoptosis of antigen-presenting cells carrying class II restricted epitopes derived from alloantigens released by a graft, from autoantigens or allergens.

### SUMMARY OF THE INVENTION

The present invention describes methods by which antigen-specific Foxp3+ regulatory T cells are elicited.

These methods comprises the general steps of:
(a) obtaining antigen-specific cytolytic CD4+ T cells;
(b) inducing apoptosis of antigen-presenting cells by exposing the antigen-presenting cells to the cytolytic CD4+ T cells;
(c) obtaining apoptotic cells and apoptotic bodies from apoptosis of the antigen-presenting cells; and
(d) incubating the apoptotic cells or the apoptotic bodies with cells capable of presenting antigens from the apoptotic cells or the apoptotic bodies.

cCD4+ T (cytolytic CD4+ cells) cells can be obtained by active immunization of an animal and prepared by affinity purification using magnetic beads coated with surface-specific antibodies. Typically CD8+, CD19+, CD127+ cell are depleted. Alternatively, the cCD4+ T cells are obtained in vitro, the method comprising the isolation of naïve CD4+ T cells. from an animal and exposure in culture to class II restricted epitopes containing a thioreductase motif within flanking residues as described herein or in WO2008/017517. Alternatively, the cCD4+ T cells can be obtained in vitro, the method comprising the isolation of polarized CD4+ T cells from an animal and exposure in culture to class II restricted epitopes containing a thioreductase motif within flanking residues as described herein or in WO2008/017517.

The cCD4+ T cells can be used in vivo to induce apoptosis of antigen-presenting cells, the method comprising the transfer of cCD4+ T cells in an animal actively producing an immune response towards the antigen recognized by cCD4+ T cells.

In embodiments of the methods of the present invention, the cCD4+ T cells are used in vitro in cultures with antigen-presenting cells presenting the epitope recognized by cCD4+ T cells to generate or obtain apoptotic bodies.

In other embodiments of the methods of the present invention, the apoptotic bodies obtained from in vitro cultures are used to load immature antigen-presenting cells and the immature antigen-presenting cells are used to generate or obtain antigen specific Tregs by cycles of stimulation using population of CD4+ T cells obtained from naïve animals.

In another aspect of the present invention, the immature antigen-presenting cells loaded with apoptotic bodies obtained from in vitro cultures are used for passive transfer into an animal in need of treatment.

In another aspect of the present invention, the immature antigen-presenting cells loaded with apoptotic bodies are used in vitro to generate or obtain Tregs, for passive transfer to an animal in need of treatment.

In a particular aspect of the present invention, the Tregs obtained (and/or isolated) by the methods described herein are used for the prevention or treatment of diseases in a subject in need for such a prevention or treatment. The disease can be an auto-immune disease, allergic disorder or a graft rejection.

An aspect of the present invention relates to in vitro methods of obtaining cells loaded with apoptotic cells or apoptotic bodies. These methods comprise the steps of:
a) providing antigen-specific cytolytic CD4+ T cells for an antigen,
b) providing antigen-presenting cells, presenting the antigen,
c) exposing the antigen-presenting cells to the cytolytic CD4+ T cells, thereby inducing apoptosis of the antigen presenting cells;
d) isolating apoptotic cells or apoptotic bodies from the antigen-presenting cells which underwent apoptosis in step c); and
e) incubating the apoptotic cells or the apoptotic bodies with cells capable of presenting antigens from the apoptotic cells or from the apoptotic bodies, thereby obtaining cells loaded with apoptotic cells or apoptotic bodies.

In addition these methods can further comprise a step f) for obtaining antigen-specific regulatory T cells by contacting the loaded cells of step e) with a further source of CD4+ cells, thereby obtaining a population of antigen-specific regulatory T cells. These cells are specific for the antigen that has been used to generate the antigen specific cytolytic CD4+ cells.

Such antigen-specific regulatory T cells are typically Foxp3 high CD4+ T cells.

According to certain embodiments, the source of CD4+ cells is selected from the group consisting of naïve CD4+ cells, polarized CD4+ T cells and natural Tregs.

According to certain embodiments, cells capable of presenting antigens from the apoptotic cells or from the apoptotic bodies are selected from the group consisting of dendritic cells, macrophages, B lymphocytes, and cells capable of expressing MHC class II determinants.

Apoptotic cells or apoptotic bodies are which are isolated in step d) can be isolated by affinity purification, centrifugation, gel filtration, magnetic beads sorting or fluorescence-activated sorting.

Cells capable of presenting antigens from apoptotic cells or apoptotic bodies can be immature antigen-presenting cells obtained by transformation of peripheral blood monocytes or bone-marrow derived precursors.

The antigens which are used in embodiments of the described methods can be an auto-immune antigen, an allergen or an antigen involved in graft rejection.

In embodiments of methods of the present invention, antigen-specific cytolytic CD4+ T cells are obtained by contacting peripheral blood cells with peptides comprising a MHC class II restricted epitope of the antigen and a sequence with the motif [CST]-X(2)-C or C-X(2)-[CST].

In embodiments of methods of the present invention antigen-specific cytolytic CD4+ T cells are obtained from naïve CD4+ T cells, polarized CD4+ T cells, or from natural Tregs.

In further embodiments of these methods further steps of isolating the cells loaded with apoptotic cells or apoptotic bodies obtained in step e) are performed.

Other embodiments of the methods of the invention comprise the step of isolating antigen-specific regulatory T cells, obtained by the described methods.

In specific embodiments, antigen-specific regulatory T cells are separated into distinct subsets based on the expression of surface markers CD25 and/or CTLA-4, which are both typically highly expressed, or on the production of cytokines TGF-beta and/or IL-10 or on the expression of Foxp3.

Another aspect of the invention relates to a population of antigen-specific Tregs obtained by the above mentioned methods.

Another aspect of the invention relates to a population of cells loaded with apoptotic cells or apoptotic bodies obtained by the above methods for use as a medicament.

Another aspect of the invention relates to a population of antigen-specific regulatory T cells obtained by the above methods for use as a medicament.

Another aspect relates to a pharmaceutical composition comprising a population of cells loaded with apoptotic cells or apoptotic bodies obtained by the above methods and a pharmaceutically acceptable carrier.

Another aspect relates to a pharmaceutical composition comprising antigen-specific regulatory T cells obtained by the above methods and a pharmaceutically acceptable carrier.

Another aspect of the present invention relates to methods of treating or preventing in a mammalian subject a disorder selected from the group of an autoimmune disease, allergic disease, graft rejection, chronic inflammatory diseases, comprising the step of administering to the mammalian subject a population of antigen-specific regulatory T cells according to claim 14 to the mammalian subject. Typically mammalian subject is a human.

Another aspect of the present invention relates to methods of treating or preventing in a mammalian subject a disorder selected from the group of an autoimmune disease, allergic disease, graft rejection, chronic inflammatory diseases, comprising the step of administering to the mammalian subject a population of cells loaded with apoptotic cells or apoptotic bodies. Typically, the mammalian subject is a human.

Diseases which can be treated with the above mentioned cells comprise autoimmune diseases, allergic diseases, graft rejection, chronic inflammatory diseases.

The autoimmune disease can be a systemic or an organ-specific autoimmune disease.

The autoimmune disease can be directed against an antigen such as thyroglobulin, thyroid peroxidase, TSH receptor, insulin (proinsulin), glutamic acid decarboxylase (GAD), tyrosine phosphatase IA-2, myelin oligodendrocyte protein and heat-shock protein HSP65.

The allergic disease against an allergen can be an allergy against an airborne allergen, food allergen, contact allergen or systemic allergen.

The graft rejection can be a graft rejection of cellular origin or of tissue origin.

Another aspect of the present invention relates to the use of antigen-specific regulatory T cells as obtained by the above methods for evaluating a mechanisms of action of antigen-specific regulatory T cells.

### BRIEF DESCRIPTION OF THE DRAWINGS

FIG. 1 shows accumulation of Foxp3+ regulatory T cells in a male skin graft carried out on a female recipient. In the graft, about 12.5% of the CD3+ lymphocyte population is represented by Foxp3+ cells, as compared to 1% in the normal skin (n=3 mice per group).

### DETAILED DESCRIPTION OF THE INVENTION

### Definitions

The term “peptide” as used herein refers to a molecule comprising an amino acid sequence of between 2 and 200 amino acids, connected by peptide bonds, but which can comprise non-amino acid structures (like for example a linking organic compound). Peptides as used in the methods of the present invention can contain any of the conventional 20 amino acids or modified versions thereof, or can contain non-naturally occurring amino acids incorporated by chemical peptide synthesis or by chemical or enzymatic modification.

The term “antigen” as used herein refers to a structure of a macromolecule, typically protein (with or without polysaccharides) or made of proteic composition comprising one or more hapten(s) and comprising T cell epitopes.

The term “antigenic protein” as used herein refers to a protein comprising one or more T cell epitopes. An auto-antigen or auto-antigenic protein as used herein refers to a human or animal protein present in the body, which elicits an immune response within the same human or animal body.

The term “food or pharmaceutical antigenic protein” refers to an antigenic protein naturally present in a food or pharmaceutical product, such as in a vaccine.

The term “epitope” refers to one or several portions (which may define a conformational epitope) of an antigenic protein which is/are specifically recognized and bound by an antibody or a portion thereof (Fab′, Fab2′, etc.) or a receptor presented at the cell surface of a B or T cell lymphocyte, and which is able, by this binding, to induce an immune response.

The term “T cell epitope” in the context of the present invention refers to a dominant, sub-dominant or minor T cell epitope, i.e. a part of an antigenic protein that is specifically recognized and bound by a receptor at the cell surface of a T lymphocyte. Whether an epitope is dominant, sub-dominant or minor depends on the immune reaction elicited against the epitope. Dominance depends on the frequency at which such epitopes are recognized by T cells and able to activate them, among all the possible T cell epitopes of a protein. In particular embodiments, a T cell epitope is an epitope recognized by MHC class II molecules, which consists of a sequence of 8 or 9 amino acids (depending on the class II haplotype) that fit in the groove of the MHC II molecule. Within a peptide sequence representing a T cell epitope, the amino acids in the epitope are numbered P1 to P9, amino acids N-terminal of the epitope are numbered P-1, P-2 and so on, amino acids C terminal of the epitope are numbered P+1, P+2 and so on.

The term “alloantigen” refers to an antigen generated by protein polymorphism in between 2 individuals of the same species.

The term “alloreactivity” refers to an immune response that is directed towards allelic differences between the graft recipient and the donor. Alloreactivity applies to antibodies and to T cells. In the context of the present invention this relates to T cell alloreactivity, which is based on T cell recognition of alloantigens presented in the context of MHC determinants as peptide-MHC complexes.

The term “major histocompatibility antigen” refers to molecules belonging to the HLA system in man (H2 in the mouse), which are divided in two general classes. MHC class I molecules are made of a single polymorphic chain containing 3 domains (alpha 1, 2 and 3), which associates with beta 2 microglobulin at cell surface. Class I molecules are encoded by 3 loci, called A, B and C in humans. Such molecules present peptides to T lymphocytes of the CD8+ subset. Class II molecules are made of 2 polymorphic chains, each containing 2 chains (alpha 1 and 2, and beta 1 and 2). These class II molecules are encoded by 3 loci, DP, DQ and DR in man.

The term “minor histocompatibility antigen” refers to peptides that are derived from normal cellular proteins and are presented by MHC belonging to the class I and/or the class II complexes. Any genetic polymorphism that qualitatively or quantitatively affects the display of such peptides at the cell surface can give rise to a minor histocompatibility antigen.

The term “homologue” as used herein with reference to the epitopes, refers to molecules having at least 50%, at least 70%, at least 80%, at least 90%, at least 95% or at least 98% amino acid sequence identity with the naturally occurring epitope, thereby maintaining the ability of the epitope to bind an antibody or cell surface receptor of a B and/or T cell. Particular homologues of an epitope correspond to the natural epitope modified in at most three, more particularly in at most 2, most particularly in one amino acid.

The term “derivative” as used herein with reference to the peptides refers to molecules which contain at least the peptide active portion (i.e. capable of eliciting cytolytic CD4+ T cell activity) and, in addition thereto comprises a complementary portion which can have different purposes such as stabilizing the peptides or altering the pharmacokinetic or pharmacodynamic properties of the peptide.

The term “organic compound having a reducing activity” refers in the context of this invention to compounds, more in particular amino acid sequences, with a reducing activity for disulfide bonds on proteins.

The term “immune disorders” or “immune diseases” refers to diseases wherein a reaction of the immune system is responsible for or sustains a malfunction or non-physiological situation in an organism. Included in immune disorders are, inter a/ia, allergic disorders and autoimmune diseases.

The terms “allergic diseases” or “allergic disorders” as used herein refer to diseases characterized by hypersensitivity reactions of the immune system to specific substances called allergens (such as pollen, stings, drugs, or food). Allergy is the ensemble of signs and symptoms observed whenever an atopic individual patient encounters an allergen to which he has been sensitized, which may result in the development of various diseases, in particular respiratory diseases and symptoms such as bronchial asthma. Various types of classifications exist and mostly allergic disorders have different names depending upon where in the mammalian body it occurs. “Hypersensitivity” is an undesirable (damaging, discomfort-producing and sometimes fatal) reaction produced in an individual upon exposure to an antigen to which it has become sensitized; “immediate hypersensitivity” depends of the production of IgE antibodies and is therefore equivalent to allergy.

The terms “autoimmune disease” or “autoimmune disorder” refer to diseases that result from an aberrant immune response of an organism against its own cells and tissues due to a failure of the organism to recognize its own constituent parts (down to the sub-molecular level) as “self”. The group of diseases can be divided in two categories, organ-specific (such as Addison disease, hemolytic or pernicious anaemia, Goodpasture syndrome, Graves disease, idiopathic thrombocytopenic purpura, insulin-dependent diabetes mellitus, juvenile diabetes, uveitis, Crohn's disease, ulcerative colitis, pemphigus, atopic dermatitis, autoimmune hepatitis, primary biliary cirrhosis, autoimmune pneumonitis, auto-immune carditis, myasthenia gravis, glomerulonephritis and spontaneous infertility) and systemic diseases such as lupus erythematosus, psoriasis, vasculitis, polymyositis, scleroderma, multiple sclerosis, ankylosing spondilytis, rheumatoid arthritis and Sjoegren syndrome). The autoimmune disorders are thus directed to own cells or tissues and include a reaction to “auto-antigens”, meaning antigens (e.g. of proteins) that are own constituent parts of the specific mammalian organism. In this mechanism, auto-antigens are recognised by B- and/or T-cells which will install an immune reaction against this auto-antigen.

A non-limitative list of diseases encompassed by the term “auto-immune diseases” or “auto-immune disorders” comprises therefore acute disseminated encephalomyelitis (ADEM), addison's disease, alopecia areata, antiphospholipid antibody syndrome (APS), Autoimmune hemolytic anemia, Autoimmune hepatitis, bullous pemphigoid, Behçet's disease, Coeliac disease, inflammatory bowel disease (IBD) (such as Crohns Disease and Ulcerative Colitis), dermatomyositis, diabetes mellitus type 1, Goodpasture's syndrome, Graves' disease, Guillain-Barré syndrome (GBS), Hashimoto's disease, Idiopathic thrombocytopenic purpura, lupus erythematosus, mixed connective tissue disease, multiple sclerosis (MS), myasthenia gravis, narcolepsy, pemphigus vulgaris, pernicious anaemia, psoriasis, psoriatic arthritis, polymyositis, primary biliary cirrhosis, rheumatoid arthritis (RA), Sjögren's syndrome, temporal arteritis, vasculitis, Wegener's granulomatosis and atopic dermatitis

An “allergen” is defined as a substance, usually a macromolecule or a proteic composition which elicits the production of IgE antibodies in predisposed, particularly genetically disposed, individuals (atopics) patients. Similar definitions are presented in Liebers et al. (1996) Clin. Exp. Allergy 26, 494-516.

The term “inflammatory diseases” or “inflammatory disorders” refers to diseases wherein the typical characteristics of inflammation are observed. This term can therefore overlap with other diseases wherein an inflammation aspect is also present. It is known in the art that a distinction can be made between “acute inflammation” and “chronic inflammatory diseases”. The term “inflammatory diseases” or “inflammatory disorders” includes but is not limited to disease selected from the group of rheumatoid arthritis, conjunctivitis, rheumatoid spondylitis, osteoarthritis, gouty arthritis, bronchitis, tuberculosis, chronic cholecystitis, inflammatory bowel disease, acute pancreatitis, sepsis, asthma, chronic obstructive pulmonary disease, dermal inflammatory disorders such as psoriasis and atopic dermatitis, systemic inflammatory response syndrome (SIRS), acute respiratory distress syndrome (ARDS), cancer-associated inflammation, reduction of tumor-associated angiogenesis, diabetes, treatment of graft v. host disease and associated tissue rejection inflammatory responses, Crohn's disease, delayed-type hypersensitivity, immune-mediated and inflammatory elements of CNS disease; e.g., Alzheimer's, Parkinson's, multiple sclerosis, etc.

The term “therapeutically effective amount” refers to a number of cells which produces the desired therapeutic or preventive effect in a patient. For example, in reference to a disease or disorder, it is the number of cells which reduces to some extent one or more symptoms of the disease or disorder, and more particularly returns to normal, either partially or completely, the physiological or biochemical parameters associated with or causative of the disease or disorder. According to one particular embodiment of the present invention, the therapeutically effective number is the number of cells which will lead to an improvement or restoration of the normal physiological situation. For instance, when used to therapeutically treat a mammal affected by an immune disorder, it is a daily number of cells per kg body weight of the mammal.

The term “natural” when referring to a peptide or a sequence herein relates to the fact that the sequence is identical to a naturally occurring sequence. This included wild-type sequences as encountered in the majority of a population, but also less frequent polymorphisms and mutations which occur in a population. In contrast therewith the term “artificial” refers to a sequence or peptide which as such does not occur in nature. Optionally, an artificial sequence is obtained from a natural sequence by limited modifications such as changing one or more amino acids within the naturally occurring sequence or by adding amino acids N- or C-terminally of a naturally occurring sequence. Amino acids are referred to herein with their full name, their three-letter abbreviation or their one letter abbreviation.

“Foxp3” (forkhead box P3) is a member of the fork-winged helix family of transcription factors, plays an important role in the development and function of naturally occurring CD4-positive/CD25-positive T regulatory cells (Tregs).

“Tregs” (regulatory T cells) Tregs are involved in active suppression of inappropriate immune responses. These cells are CD4+, CD25+, FoxP3+ cells.

“Naïve cd4+ T cells” express CD62L, have a low or intermediate expression of CD44, and a low cytokine production with no preferred pathway.

“Polarised CD4+ cells” have a high CD44 expression and the production of a restricted set of cytokines.

“cCD4+ T cells” Cytolytic CD4+ T cells have been characterised in detail in WO 2009/101207 and are characterised by one or more of the following properties:
Undetectable expression of the transcription repressor Foxp3,
An increased activity of the serine-threonine kinase AKT, compared to natural CD4+ regulatory T-cells,
Undetectable production of TGF-beta and undetectable or very low production of IL-10 when compared to natural CD4+ regulatory T-cells,
High concentrations of IFN-gamma, when compared to natural CD4+ regulatory T-cells,
Co-expression of the transcription activators T-bet and GATA3 after antigenic stimulation,
Expression of NKG2D,
Production of high concentrations of soluble Fas ligand (FasI).

### DESCRIPTION OF THE INVENTION

The general principle of the present invention relates to the use of apoptotic bodies obtained from specific antigen-loaded antigen-presenting cells to elicit the production of antigen-specific Foxp3+ Tregs. Specifically, the purpose of the present invention is to provide methods by which selective apoptosis is obtained from antigen-presenting cells presenting an autoantigen or an antigen to which an immune response is undesirable (e.g. an allergen, an alloantigen from a graft or an allofactor used of therapeutic purposes), in the context of MHC class II determinants. As described in greater detail below, the present invention therefore provides methods of generating, obtaining or isolating antigen-specific Tregs, thereby providing the possibility of switching off an immune response specific for a given antigen.

Cells induced in apoptosis proceed through a number of surface alterations, including oxidation of phosphatidylserine, polysaccharides, and glycolipids, which render them recognizable by phagocytes. In addition, apoptotic cells express new proteins, such as thrombospondin-1 and/or localize at their surface intracellular components such as phosphatidylserine, DNA and nucleosomes. Altogether these surface alterations provide a possibility for phagocytes to engulf apoptotic cells without triggering an innate immune reaction, in the absence of ligation by innate receptors such as Toll-like receptors, NOD or RIG.

Phagocytic cells removing apoptotic cells are equipped with recognition receptors, such as CD14, scavenger receptors and C-type lectin receptors (Ravichandran & Lorenz (2007) Nature Rev. Immunol. 7, 964-974). Scavenger receptors are surface glycoproteins able to bind oxidized or acetylated low-density lipoproteins (LDL) as well as polyanionic ligands and apoptotic cells. Examples of scavenger receptors include CD36, LOX-1 and CLA-1. Recognition is followed by rapid internalization and, in the case of apoptotic cells, destruction and fusion with endosomes and lysosomes. Of particular interest is the production of thrombospondin-1 by apoptotic cells, which acts as a soluble bridge with CD36 expressed on phagocytes. Expression of thrombospondin-1 is caspase-dependent.

Soluble factors also play a role in the removal of apoptotic cells. Examples include collectins and collectin-like molecules, such as mannose binding lectin and C1q. Both interact with calreticulin expressed at the surface of phagocytes. The family of pentraxins, which include serum amyloid P (SAP) and C-reactive protein (CRP) and prototypic pentraxin (PTX) also bind to apoptotic cells.

Altogether, there are a large number of factors that are used under physiological conditions to dispose of cells undergoing natural programmed cell death, or apoptosis (Jeannin et al. (2008) Curr. Opinion in Immunol. 20, 530-537). In mammals, there is a constant renewal of cells to maintain normal cell numbers and activity (Steinman et al. (2000) J. Exp. Med. 191, 411-416). In the absence of inflammatory conditions, apoptotic bodies are taken up in organs by antigen-presenting cells, which migrate towards regional lymph nodes in which an exchange of apoptotic bodies occurs with lymph node dendritic cells, either directly or as a consequence of the rapid lysis of the migrating antigen-presenting cells. At least some of the dendritic cells migrating to regional lymph nodes are found to be immature, which exhibit a high capacity to phagocyte apoptotic cells. In the lymph node, dendritic cells are primarily in an immature status, but seemingly belong to a subset showing the capacity to present antigens in both class I and class II determinants. In the absence of co-stimulatory signals related to the non-inflammatory conditions, MHC class II presentation provides the recruitment and activation signals required for Tregs. Such Tregs are antigen-specific (directed towards autoantigens) and suppress activation of a response towards such autoantigens. Thus, apoptosis occurring in a non-inflammatory context elicits antigen-specific Tregs, which maintain tolerance to self-antigens.

Conversely, under inflammatory conditions, as it occurs in autoimmune diseases or responses to alloantigens or allergens or during an immune response elaborated against infectious agents, there is an increase in the production of apoptotic bodies, which are carried to regional lymph nodes. This massive influx of cells loaded with apoptotic bodies exceeds the capacity of lymph node dendritic cells to capture such bodies to present them in order to recruit and activate Tregs. Additionally, the presence of pro-inflammatory cytokines alters the phenotype of lymph node dendritic cells, which are induced into maturation and, consequently, increases activation of effector T cells to the detriment of Tregs. Although this is a desirable effect during a response to infectious agents, in the context of autoimmune diseases, allergic reactions, and graft rejection, it unfortunately leads to further tissue destruction and inflammation. It would therefore be advantageous to devise a novel method to increase the capacity to generate or obtain apoptotic bodies in a non-inflammatory context to generate, obtain or isolate antigen-specific Tregs.

During (yet unpublished) studies on the elicitation of cCD4+ T cells using MHC class II restricted epitopes carrying a thioreductase motif within flanking residues, it was unexpectedly found that a consequence of the induction of cCD4+ was an accumulation of Foxp3+ Tregs in target organs. Thus, in a model of skin graft rejection, the long-term persistence of an allogeneic graft was accompanied by the presence of Foxp3+ Tregs in the graft itself. The same observation was made in experimental models of multiple sclerosis, in which prevention or suppression of diseases was accompanied by accumulation of Foxp3+ Tregs in central nervous system (CNS) white matter.

The methods of the present invention therefore also comprise in a particular embodiment the use of MHC class II restricted epitopes carrying a thioreductase motif within flanking residues as described in WO2008/017517 (which is included herein by reference).

In general, the peptides used in embodiments of the present invention are peptides which comprise at least one T-cell epitope of an antigen (self or non-self) with a potential to trigger an immune reaction, coupled to an organic compound having a reducing activity, such as a thioreductase sequence motif [CST]-X(2)-[CST] wherein at least one of [CST] is Cys; thus the motif is either [C]-X(2)-[CST] or [CST]-X(2)-[C]. In particular embodiments peptides contain the sequence motif [C]-X(2)-[CS] or [CS]-X(2)-[C]. In more particular embodiments peptides contain the sequence motif C-X(2)-S, S-X(2)-C or C-X(2)-C. The T cell epitope and the organic compound are optionally separated by a linker sequence.

These peptides can be made by chemical synthesis, which allows the incorporation of non-natural amino acids. Accordingly, in the motif of reducing compounds C represents either cysteine or other amino acids with a thiol group such as mercaptovaline, homocysteine or other natural or non-natural amino acids with a thiol function. In order to have reducing activity, the cysteines present in the motif should not occur as part of a cystine disulfide bridge, or as oxidised cysteines. Nevertheless, the motif may comprise modified cysteines such as methylated cysteine, which is converted into cysteine with free thiol groups in vivo.

The amino acid X in the [CST]-X(2)-[CST] motif of the reducing compounds can be any natural amino acid, including S, C, or T or can be a non-natural amino acid. In particular X is an amino acid with a small side chain such as Gly, Gly, Ser or Thr. Alternatively is not an amino acid with a bulky side chain such as Tyr. Morer particularly, at least one X in the [CST]-X(2)-[CST] motif is His or Pro.

In the peptides comprising the motif described above as the reducing compound, the motif is located such that, when the epitope fits into the MHC groove, the motif remains outside of the MHC binding groove. The motif is placed either immediately adjacent to the epitope sequence within the peptide, or is separated from the T cell epitope by a linker. More particularly, the linker comprises an amino acid sequence of 7 amino acids or less. Most particularly, the linker comprises 1, 2, 3, or 4 amino acids. Alternatively, a linker comprises 5, 6, 7, 8, 9 or 10 amino acids. In those peptides where the motif sequence is adjacent to the epitope sequence this is indicated as position P-4 to P-1 or P+1 to P+4 compared to the epitope sequence.

In addition to the reducing motif, such peptides comprise (as described in the prior art), a T cell epitope derived from an antigen, typically an allergen or an auto-antigen, depending on the application. Such a T cell epitope in a protein sequence can be identified by functional assays and/or one or more in silico prediction assays.

Suitable algorithms are described for example in Zhang et al. (2005) Nucleic Acids Res 33, W180-W183 (PREDBALB); Salomon & Flower (2006) BMC Bioinformatics 7, 501 (MHCBN); Schuler et al. (2007) Methods Mol. Biol. 409, 75-93 (SYFPEITHI); Donnes & Kohlbacher (2006) Nucleic Acids Res. 34, W194-W197 (SVMHC); Kolaskar & Tongaonkar (1990) FEBS Lett. 276, 172-174 and Guan et al. (2003) Appl Bioinformatics 2, 63-66 (MHCPred).

The amino acids in a T cell epitope sequence are numbered according to their position in the binding groove of the MHC proteins. Typically the T-cell epitope present within the peptides consists of between 8 and 25 amino acids, yet more particularly of between 8 and 16 amino acids, yet most particularly consists of 8, 9, 10, 11, 12, 13, 14, 15 or 16 amino acids.

More particularly, the T cell epitope consists of a sequence of 9 or 8 amino acids. More particularly, the T-cell epitope is an epitope, which is presented to T cells by MHC-class II molecules. Especially the T cell epitope sequence is an epitope sequence which fits into the cleft of an MHC II protein, more particularly a nonapeptide fitting into the MHC II cleft.

The T cell epitope of a peptide can correspond either to a natural epitope sequence of a protein or can be a modified version thereof, provided the modified T cell epitope retains its ability to bind within the MHC cleft, similar to the natural T cell epitope sequence. The modified T cell epitope can have the same binding affinity for the MHC protein as the natural epitope, but can also have a lowered affinity. The binding affinity of the modified peptide is in such cases no less than 10-fold less than the original peptide, more particularly no less than 5 times less.

Examples of (auto-)antigens from which the T-cell epitopes can be derived for use in embodiments of methods of the invention are thyroglobulin, thyroid peroxidase, TSH receptor, insulin (proinsulin), glutamic acid decarboxylase (GAD), tyrosine phosphatase IA-2, myelin oligodendrocyte protein and heat-shock protein HSP65.

Without intending to be limiting and being bound by theory, the general mechanism of action comprises the following steps:
(a) Immature dendritic cells loaded with apoptotic bodies elicit Tregs specific for determinants presented in MHC class II determinants.
(b) These Tregs migrate to the location in which there is an unwanted immune response.
(c) The accumulation of these Tregs in the target location results in a control of inflammation as a consequence of the numerous anti-inflammatory properties of the Tregs
(d) Tissue destruction and the production of apoptotic cells is suppressed and normal cell turnover in the target location is re-established thereby restoring normal tissue function

The present invention provides various embodiments of methods by which antigen-specific Tregs can be obtained. In an embodiment of methods of the invention, apoptosis of antigen-presenting cells may be obtained in vitro by exposure to cCD4+ T cells. Apoptotic bodies are used to load immature dendritic cells. The immature dendritic cells loaded with apoptotic bodies are either used for cell therapy or used in vitro for generating, isolating or obtaining antigen-specific Tregs, for use in cell therapy. Cell therapy in the context of the present invention comprises the step of preparing cells for administration to a mammal.

A general method for inducing of apoptosis of cells in vitro is described and known in the art. For example, apoptosis of CD4+ T cells lymphocytes can be obtained by culturing them in the presence of insolubilized antibodies to CD3 and CD28. The methods used to determine whether cells are actually apoptotic are well described in the art. These methods include the binding of annexin V on phospholipids expressed at the surface of apoptotic cells, activation of caspases, and degradation of nucleic acid. Reviews on these methods can be found in publications such as in Fuchs and Steller (2011), Cell 147, 742-758.

In the present invention, and unlike the prior art, apoptosis induced in antigen-presenting cells requires the formation of a synapse between the antigen-presenting cell (APC) and the cell inducing apoptosis, i.e. cCD4+ T cells. The formation of a synapse activates the cytolytic properties of the cCD4+ T cell, resulting in induction of apoptosis only of the cells presenting the corresponding antigen-derived class II-restricted epitopes. Advantageously, this provides strict antigen specificity. In the absence of any additional reagent for the assay system, such as anti-CD3 antibodies, the in vitro induction of apoptosis described in methods of the present invention reproduces conditions close to those occurring in vivo.

Apoptosis of antigen-presenting cells by CD4+ T cells has been reported by Janssens et al. (2008) J. Immunol. 171, 4604-4612). Tregs, under some circumstances, could induce target cell apoptosis and a number of mechanisms of induction have been described, including activation of IDO release of granzyme B with or without perforin. A review of these mechanisms can be found in (Shevach (2011) Adv. Immunol. 112, 137-176). In the methods of the present invention, the cCD4+ T cells represent a unique cell subset, distinct from Tregs on both phenotypic and functional properties. The methods by which such cCD4+ T cells can be induced can be found in WO2008/017517.

Methods for the identification and isolation of apoptotic bodies are known in the art. Apoptotic cells or apoptotic bodies express a number of novel constituents at their surface and can, in addition, be opsonized by soluble factors, as described above. These two types of alterations provide ways to isolate apoptotic cells or apoptotic bodies. Examples of this can be found in the art (Schiller et al. (2008) Cell Death Diff. 15, 183-191). One example is the use of an antibody to thrombospondin to isolate cells or cell debris, which, because of entering into an apoptotic cycles, express thrombospondin.

In an embodiment of the present invention, isolated apoptotic bodies or apoptotic cells are incubated with dendritic cells to allow engulfment, processing, and presentation in the context of MHC class II determinants. Different subsets of dendritic cells have been described, varying in function, surface phenotype, and maturity. In general, an immature dendritic cell has a high capacity to take up apoptotic cells and apoptotic bodies, but may not be efficient in terms of expression of epitopes within MHC class II determinants. However, some subsets of dendritic cells, in particular those housed within lymph nodes, combine the two properties, uptake of apoptotic cells or apoptotic bodies and presentation of epitopes at their surface.

In the context of the present invention, however, dendritic cells are derived in vitro and kept immature by methods well described in the art. The prior art teaches that derivatization of dendritic cells in the presence of interferon-gamma (IFN-gamma) induces a highly mature status, whereas IL-4 will maintain dendritic cells in an immature status. Dendritic cells can be derived from either peripheral blood monocytes or from bone marrow precursors. Apoptotic cells and apoptotic bodies obtained as described above are incubated with immature dendritic cells, thereby allowing presentation by MHC class II determinants.

It should be clear to one skilled in the art that dendritic cells are a preferred, but not exclusive means to obtain presenting cells capable of presenting antigens processed from apoptotic cells or apoptotic bodies. Alternatives include but are not limited to macrophages, endothelial, or epithelial cells, which can be induced in MHC class II expression.

Another aspect of the inventions relates to the use in cell therapy of dendritic cells loaded with antigens derived from apoptotic cells or apoptotic bodies. By way of example, dendritic cells presenting class II restricted epitopes derived from apoptotic bodies obtained by the cytolytic action of cCD4+ T cells on antigen-presenting cells presenting an autoantigen are administered intravenously to animals affected by a disease process in which an immune response to the autoantigen is implicated. The result of such cell therapy is the specific suppression of the immune response and the cure of the disease. Additional examples are provided below, but the scope of the present invention is not restricted to such examples.

In a specific embodiment of the methods of the present invention, immature dendritic cells loaded with apoptotic cells or apoptotic bodies are maintained in culture to which a population of CD4+ T cells is added for incubation to generate, isolate or obtain Tregs. Several possible sources of CD4+ T cells can be used, including but not limited to: cells obtained from naïve animals and prepared by affinity using, for instance, magnetic beads coated with specific antibodies; polarized CD4+ T cells obtained from the spleen, lymph nodes, tissues, or peripheral blood from animals in which a disease process is ongoing related to an immune response to the (auto)antigen to which it is desirable to elicit Tregs; or natural Tregs, as defined as showing high and stable expression of the Foxp3 repressor of transcription.

It should be clear to one skilled in the art that each one of these 3 sources of CD4+ T cells may be more appropriate than the others, given relevant circumstances. By way of example, naïve CD4+ T cells are easily accessible even from peripheral blood and provide a repertoire, which is large enough to recognize any antigen. In situations in which it is preferred to prevent a disease process, and thereby in which the antigen can be chosen according, inter alia, to the MHC class II haplotype of a given animal, naïve CD4+ T cells would represent the best choice. On the other hand, polarized CD4+ T cells represent a source of cells for the practice of the methods of the present invention in situations in which it is preferred to use cells with increased affinity for peptide-MHC complexes. One example is provided by autoreactive CD4+ T cells found in type 1 diabetes, in which the recognition of insulin-derived peptides by CD4+ T cells occurs primarily through incomplete binding to peptide-MHC complexes, resulting in a relatively low T cell receptor affinity.

In the present invention, one embodiment is the use of natural Tregs as a source. The repertoire of Tregs is shaped towards recognition of self-antigens and, as described above, such cells have a sufficient affinity to functionally form synapse with antigen-presenting cells. The number of antigen-specific natural Tregs towards a given antigen is exceedingly low as such Tregs represent only 5 to 10% of the total CD4+ T cell number. The present invention provides methods by which such low numbers can be increased in vitro. A further advantage of using natural Tregs, which are thymus selected, in the methods of the present invention is their reported phenotypic stability. Thus, in natural Tregs, expression of Foxp3 is high and remains stable over time and under various activation conditions. By contrast, Tregs induced into the periphery and acquiring Foxp3 expression may be unstable, due to the absence of epigenomic signature of commitment to the Treg lineage and loose their regulatory properties when the context changes in which they are active, as for instance under inflammatory conditions.

In another aspect of the invention, Tregs expanded (natural Tregs) or induced (naïve or polarized) by in vitro culture with immature dendritic cells presenting antigens derived from apoptotic cells or apoptotic bodies are used for cell therapy. Such therapy can be administered as a preventive therapy, as for example in the prevention of graft rejection, or as a suppressive therapy, as for instance in type 1 diabetes.

Optionally, Tregs obtained by methods described in the present invention can be further expanded using non-specific means when it is desired to further increase the number of such Tregs. Examples of such non-specific methods are known in the art. For instance, cells incubated in the presence of insolubilized anti-CD3 and anti-CD28 antibodies and IL-2 can be expanded by several orders of magnitude.

It should be clear to one skilled in the art that, prior to cell administration, further steps could be added. One possibility is to further restrict the specificity of Tregs by incubating cells with tetramers of MHC class II determinants loaded with one or more of a synthetic peptide to which it is desirable to orientate Tregs. Another possibility is to sort out cells by a surface marker or various degree of Foxp3 expression. A population of cells with particularly high expression of Foxp3 is known to be part of the whole natural Treg population and present characteristics which make them particularly suitable in the context of the present invention.

In another aspect of the invention, Tregs obtained by the methods of the present invention can be used to establish the relevance of a given antigen or epitope for the development of a disease process. In many diseases, there is more than one antigen involved in the process, yet it remains difficult to identify the most important one. Producing antigen-specific Tregs by practicing the methods of the present invention provides a method to switch off specific antigens as a means to isolate and identify the role of specific antigens in the development of disease.

Antigen-specific Tregs obtained by the methods of the present invention provide a method to determine the importance of the Treg phenotype in its function. As an example, antigen-specific Tregs are sorted according to expression of granzyme and populations of granzyme+ and granzyme(−) are compared in terms of capacity to suppress a response either in vitro or in vivo.

The various aspects and embodiments of the present invention are illustrated in the following examples. There is, however, no intention to restrict the scope of the invention to such examples.

### Examples

### Example 1

### Induction of Apoptosis In Vitro

Antigen-presenting cells (APC) are prepared from C57BL/6 mice and loaded with a peptide encompassing a class II-restricted T cell epitope of an autoantigen implicated in experimental autoimmune encephalomyelitis (EAE), used as a model of multiple sclerosis.

Thus, a Myelin Oligodendrocyte Glycoprotein (MOG) peptide of sequence VGWYRSPFSRVVHLYR [SEQ ID. NO: 1], which corresponds to amino acid residues 37-52 of the MOG protein, is used to load cells.

This peptide contains a dominant T cell epitope. The P1 position, i.e. the first amino acid anchored into the MHC class II groove is Y40 (the P1-P9 sequence is underlined).

Cytolytic CD4+ T cells (cCD4+ T cells) are obtained from the spleen of animals immunized 4 times, using aluminium hydroxyde, with 50 μg of a peptide of SEQ ID. NO:1 in which the 3 amino acids of the amino terminal end of the peptide are replaced by the sequence CGPC, resulting in the peptide of sequence CGPCYRSPFSRVVHLYR [SEQ ID. NO: 2].

cCD4+ T cells are cultured in the presence of loaded APC overnight at 37° C., cells are washed and the extent of APC apoptosis is measured using an antibody against activated caspase 3.

### Example 2

### Isolation of Apoptotic Bodies

The supernatants of the apoptotic cells obtained in Example 1 are collected and submitted to two centrifugation steps (500×g, 5 min) to remove cells. The supernatants were then filtered though a 1.2 μM hydrophilic syringe filter. After centrifugation at 100,000×g for 30 minutes, apoptotic bodies contained in the pellet are harvested and used for cell experiments.

Alternatively, apoptotic cells and apoptotic bodies can be isolated by affinity using antibodies against cell surface components expressed as a result of apoptosis. An example of these are anti-thrombospondin antibodies. In a preferred preparation step, anti-thrombospondin antibodies are covalently coupled to magnetic microbeads. After incubation with gentle shaking for 1 h at 20° C., magnetic beads are retained on a magnet. Apoptotic bodies are then recovered by elution with slightly acidic buffer.

These methods are described in the prior art. (Schiller et al. (2008) Cell Death Diff. 15, 183-191; Gautier et al. (1999) J. Immunol. Methods 228, 49-58)

### Example 3

### Generation of or Obtaining Immature Dendritic Cells (iDC)

Bone marrow progenitor cells are obtained from upper and lower knee bones. B and T lymphocytes are removed by magnetic depletion with CD19 and CD90 microbeads, respectively. The negative fraction containing the CD19− CD90− iDC progenitors is resuspended in serum free medium containing 500 U/ml recombinant GM-CSF and seeded (3×10⁶ cells/ml) on tissue culture plates and kept at 37° C. Cells are washed every other day for 6 days, avoiding breaking the aggregates. On day 6, iDC aggregates are removed, washed and added to a new plate. On day 7, cells are harvested and used in assays.

These methods are described in the prior art. See for instance Inaba et al. (2009) Curr. Prot. immunol. 1(86), unit 3.7. p 10-12.

### Example 4

### Generation of or Obtaining Antigen-Loaded Immature Dendritic Cells

iDC show a high capacity to engulf apoptotic bodies. Therefore, iDC as obtained in Example 3 are incubated with apoptotic bodies as obtained in Example 2. For this, 2×10⁵ iDC are plated in microculture wells by an incubation of 30 min at 37° C. A suspension of apoptotic bodies is then added to the culture for a further incubation of 16 h at 37° C. Cells are then washed and resuspended in medium.

### Example 5

### Use of Antigen-Loaded Dendritic Cells for Cell Therapy

iDC loaded with apoptotic bodies are injected (2×10⁵) by the intravenous route into animals prior to or after disease induction.

Thus, C57BL/6 mice are submitted to a protocol including administration of the MOG peptide (see example 1 for the peptide of SEQ ID: NO1) in complete Freund's adjuvant with a mycobacterium extract, and 2 injections of pertussis toxin. This protocol elicits the development of signs comparable to human multiple sclerosis within 2 weeks after MOG peptide administration.

In such a model, iDC loaded with apoptotic bodies, as obtained in Example 4, are injected to mice either one day prior to disease induction or after the first signs of disease are observed, namely 2 weeks after induction.

Animals in which no iDC are injected, or animals in which unloaded iDC are injected are used as controls. The prevention or suppression of disease signs is evaluated in the experimental group and in the two control groups.

### Example 6

### Use of Antigen-Loaded Dendritic Cells to Elicit Antigen-Specific Tregs

iDC loaded with apoptotic bodies allow to generate Tregs in vitro.

Thus, iDC as described in Example 4 are maintained in culture.

T cells are isolated from the spleen of naïve mice by magnetic microbead sorting using antibodies to deplete CD8+, CD19+, CD127+ cells, followed by positive selection of CD25+ cells. The percentage of CD4+Foxp3^{high} cells is checked by fluorescence-activated cell sorting (facs) using a Foxp3 specific antibody after cell permeation. Prior art discloses the method to obtain such cells (Peters et al. (2003) Plos one 3, 574-584). Cell purity above 85% is obtained.

CD4+Foxp3^{high} cells are then added (1×10⁶ cells per well) to cultures of iDCs as described in Example 4. After a stimulation cycle of 7 days at 37° C., in the presence of IL-2 (20 IU/ml), cells are washed and re-incubated according to the same protocol using a fresh batch of iDC loaded with apoptotic bodies. After this second cycle of stimulation, cells can optionally be further expanded by incubation with magnetic beads coated with anti-CD3 and anti-CD28 antibodies in the presence of IL-2. Cells are washed and evaluated by facs for expression of Foxp3.

### Example 7

### Use of Antigen-Specific Tregs for Cell Therapy

Cells as prepared in Example 6 can be used for passive administration in the context of an autoimmune disease.

Thus, a protocol similar to the one described in Example 5 is followed but including IV administration of 2×10⁵ CD4+Foxp3^{high} cells instead of iDC.

It is shown that, as compared to control animals in which no CD4+Foxp3^{high} cells are injected, there is a significant prevention and/or suppression of disease signs.

### Example 8

### Sorting Out of Antigen-Specific Tregs for Analytical Purposes

The population of CD4+Foxp3^{high} cells can be further analyzed to determine the importance of single component or combination of components for their mechanism of action.

Thus, CD4+Foxp3^{high} cells are separated using magnetic microbeads coated with an antibody against FasL. The two populations of cells, FasL+ and FasL(−), are then tested functionally and compared for their capacity to elicit tolerance. This is carried out using an assay system in which polyclonal effector CD4+ lymphocytes, characterized by a CD4+CD25(−) phenotype, are isolated from the spleen of a naïve animal.

Natural Tregs are usually defined by their capacity to exert bystander suppression on effector cells. The assay system used here involves activation of the CD4+CD25+ T cell population by non-specific stimulation, namely a combination of anti-CD3 and anti-CD28 antibodies.

The capacity of FasL+CD4+Foxp3^{high} cells to suppress the proliferation of CD4+CD25(−) T cells is compared to that of FasL(−)CD4+Foxp3^{high} cells.

It is shown that cells expressing FasL show a higher capacity to suppress effector cell proliferation.

### Example 9

### Accumulation of Foxp3 Tregs in Skin Grafts

C57BL/6 female mice, 8 to 10 weeks old, were immunized by injecting 50 μg of a peptide encompassing a class II-restricted epitope of Dby containing a thioredox motif within flanking residues (ccDby) prior to skin grafting, as described in WO 2009/100505.

Full-thickness skin of a syngeneic male donor was grafted on the back of the recipient and followed for signs of rejection. All mice tolerated the graft. A biopsy of the graft was taken 6 weeks after grafting for histological analysis. It is shown that Foxp3+ cells accumulate in the graft.

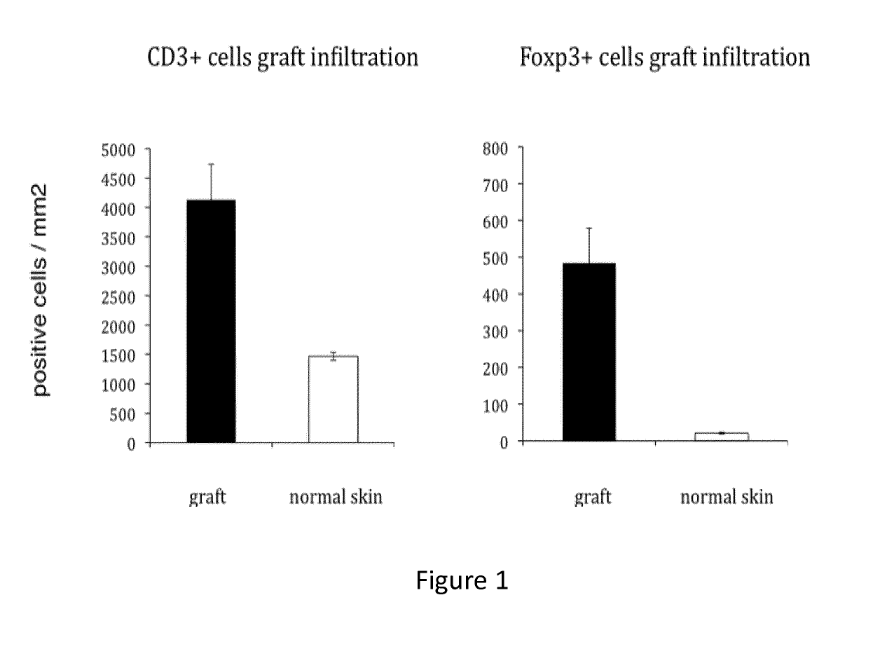

As a comparison, the content in Foxp3+ T cells in a normal skin is shown in the FIG. 1.

1. An in vitro method of obtaining antigen presenting cells loaded with apoptotic cells or apoptotic bodies, said method comprising the steps of:
a) providing antigen-specific cytolytic CD4+ T cells for an antigen,
b) providing antigen-presenting cells capable of expressing MHC class II determinants, presenting said antigen,
c) exposing said antigen-presenting cells to said cytolytic CD4+ T cells, thereby inducing apoptosis of said antigen presenting cells;
d) isolating apoptotic cells or apoptotic bodies from the antigen-presenting cells which underwent apoptosis in step c); and
e) incubating said apoptotic cells or said apoptotic bodies with cells capable of presenting antigens,.

2. The method according to claim 1, further comprising the step of isolating said cells loaded with apoptotic cells or apoptotic bodies obtained in step e).

3. A method for obtaining antigen-specific natural regulatory T cells comprising the steps of:
a) providing antigen-specific cytolytic CD4+ T cells for an antigen,
b) providing antigen-presenting cells capable of expressing MHC class II determinants, presenting said antigen,
c) exposing said antigen-presenting cells to said cytolytic CD4+ T cells, thereby inducing apoptosis of said antigen presenting cells;
d) isolating apoptotic cells or apoptotic bodies from the antigen-presenting cells which underwent apoptosis in step c); and
e) incubating said apoptotic cells or said apoptotic bodies with cells capable of presenting antigens, f) contacting said loaded cells of step e) with a population of cells comprising natural regulatory T cells, thereby increasing the number of antigen-specific regulatory T cells.

4. The method according to claim 3, further comprising the step of isolating said antigen-specific regulatory T cells.

5. The method according to claim 4, further comprising the step of separating said antigen-specific regulatory T cells into distinct subsets based on the expression of surface markers CD25 and/or CTLA-4, or on the production of cytokines TGF-beta and/or IL-10 or on the expression of Foxp3.

6. The method according to any one of claims 3 to 5, wherein said antigen-specific regulatory T cells are Foxp3 high CD4+ T cells.

7. The method according to any one of claims 1 to 6, wherein in step e) said cells capable of presenting antigens are selected from the group consisting of dendritic cells, macrophages, B lymphocytes, and cells capable of expressing MHC class II determinants.

8. The method according to any one of claims 1 to 7, wherein apoptotic cells or apoptotic bodies are isolated in step d) by affinity purification, centrifugation, gel filtration, magnetic beads sorting or fluorescence-activated sorting.

9. The method according to any one of claims 1 to 8, wherein in step e) said cells capable of presenting antigens are selected from the group consisting of immature antigen-presenting cells obtained by transformation of peripheral blood monocytes or bone-marrow derived precursors.

10. The method according to any one of claims 1 to 9, wherein said antigen in step a) is an auto-immune antigen, an allergen or an antigen involved in graft rejection.

11. The method according to any one of claims 1 to 10, wherein in step a) said antigen-specific cytolytic CD4+ T cells are obtained by contacting peripheral blood cells with peptides comprising a MHC class II restricted epitope of said antigen and a sequence with the motif [CST]-X(2)-C or C-X(2)-[CST].

12. The method according to any one of claims 1 to 11, wherein in step a) said antigen-specific cytolytic CD4+ T cells are obtained from naïve CD4+ T cells, polarized CD4+ T cells, or from natural Tregs.

13. A population of antigen-specific Tregs obtained by any one of claims 3 to 12.

14. A population of antigen presenting cells loaded with apoptotic cells or apoptotic bodies from a cell presenting a specific antigen obtained by claim 1, 2 or any one of claims 6 to 12.

15. A population of antigen-specific regulatory T cells according to claim 13 for use as a medicament.

16. A population of antigen-specific regulatory T cells according to claim 13 for use in the treatment or prevention of an autoimmune diseases, allergic disease, graft rejection, or chronic inflammatory diseases.

17. The population of antigen-specific regulatory T cells according to claim 13 for use in the treatment or prevention of a systemic or an organ-specific autoimmune diseases.

18. The population of antigen-specific regulatory T cells according to claim 13 for use in the treatment or prevention of an autoimmune disease against an antigen selected from the group of antigens consisting of thyroglobulin, thyroid peroxidase, TSH receptor, insulin (proinsulin), glutamic acid decarboxylase (GAD), tyrosine phosphatase IA-2, myelin oligodendrocyte protein and heat-shock protein HSP65.

19. The population of antigen-specific regulatory T cells according to claim 13 for use in the treatment or prevention of an allergic disease against an allergen selected from the group of antigens consisting of airborne allergens, food allergens, contact allergens and systemic allergens.

20. The population of antigen-specific regulatory T cells according to claim 13, for use in the treatment or prevention of a graft rejection of cellular origin or of tissue origin.

21. Use of antigen-specific regulatory T cells of claim 13 for evaluating a mechanism of action of said antigen-specific regulatory T cells.

22. A population of cells loaded with apoptotic cells or apoptotic bodies according to claim 14 for use in the treatment or prevention of an autoimmune diseases, allergic disease, graft rejection or chronic inflammatory diseases.

23. The population of cells loaded with apoptotic cells or apoptotic bodies according to claim 14 for use in the treatment or prevention of a systemic or an organ-specific autoimmune diseases.

24. The population of cells loaded with apoptotic cells or apoptotic bodies according to claim 14 for use in the treatment or prevention of an autoimmune disease against an antigen selected from the group of antigens consisting of thyroglobulin, thyroid peroxidase, TSH receptor, insulin (proinsulin), glutamic acid decarboxylase (GAD), tyrosine phosphatase IA-2, myelin oligodendrocyte protein and heat-shock protein HSP65.

25. The population cells loaded with apoptotic cells or apoptotic bodies according to claim 14, for use in the treatment or prevention of an allergic disease against an allergen selected from the group of antigens consisting of an airborne allergen, food allergen, contact allergen and systemic allergen.

26. The population of cells loaded with apoptotic cells or apoptotic bodies according to claim 14, for use in the treatment or prevention of a graft rejection of cellular origin or of tissue origin.

27. A method of treating or preventing in a mammalian subject a disorder selected from the group of an autoimmune disease, allergic disease, graft rejection, chronic inflammatory diseases, comprising the step of administering to said mammalian subject a population of antigen-specific regulatory T cells according to claim 13.

28. A method of treating or preventing in a mammalian subject a disorder selected from the group of an autoimmune disease, allergic disease, graft rejection, chronic inflammatory diseases, comprising the step of administering to said mammalian subject a population of cells loaded with apoptotic cells or apoptotic bodies according to claim 14.


SEQUENCE LISTING (2 sequences)

SEQ ID NO: 1 - protein, 16 aa, Artificial Sequence
  T cell epitope of MOG protein
VGWYRSPFSRVVHLYR

SEQ ID NO: 2 - protein, 17 aa, Artificial Sequence
  modified sequence of MOG epitope
CGPCYRSPFSRVVHLYR